(19) United States
(12) Patent Application Publication
(10) Pub. No.: US 2021/0366622 A1
(43) Pub. Date: Nov. 25, 2021

(54) METHODS FOR CONTROL OF PANDEMICS BY WIDESPREAD GATHERED DATA FROM ACCESSIBLE TOOLS

(71) Applicant: Thermoguard Ltd, Kiryal Ono (IL)

(72) Inventors: Nir ZAKAY, Kiryat Ono (IL); Liat ZAKAY, Givataim (IL); Offir REMEZ, Ramat Gan (IL)

(73) Assignee: Thermoguard Ltd, Kiryat Ono (IL)

(21) Appl. No.: 17/329,440

(22) Filed: May 25, 2021

Related U.S. Application Data
(60) Provisional application No. 63/029,635, filed on May 25, 2020.

(51) Int. Cl. G16H 50/80; A61B 5/00; G16H 50/70; G16H 50/20

(52) U.S. Cl. CPC G16H 50/80; A61B 5/0008; G16H 10/40; G16H 50/20; G16H 50/70

(57) ABSTRACT

The invention is a method to analyze and control the spread of viral, bacterial, fungal or other infections in the form of a pandemic by collecting and analyzing data from different wide spread sources. The use of communications, a high number of sensors, the ability to geolocate and geotag measurements, and tools for trend analysis and prediction of large data amounts, allows to detect trends, green-light areas that are not infected, contain infected areas, generate recommendations, analyze the time that it takes to develop symptoms in different demographics and more.

### RELATED APPLICATION(S)

This application claims the benefit of priority under 35 USC § 119(e) of U.S. Provisional Patent Application No. 63/029,635 filed on May 25, 2020, the contents of which are all incorporated by reference as if fully set forth herein in their entirety.

### FIELD AND BACKGROUND OF THE INVENTION

The invention herein relates to the field of pandemics control, meaning the methods, tools and analyses used to detect and control an epidemic or a pandemic. More specifically, it relates to the use of mass gathered real time information from a multiplicity of sensors and sources to assess a pandemics status and aid in decision-making.

Prior art has taught devices and methods for detecting symptoms of diseases, and the use of such sensors to assess whether patients or other subjects are sick or carrying an infectious pathogen. Prior art has also taught the use of such devices in conjunction with local communications for the sake of data transfer to other local devices such as mobile phones and computers.

Further prior art has shown some use of sensors for the sake of pandemics control. In [1] thermometers are used in conjunction with geographic data for said purposes. [2] and [3] show the use of different sensors for application vis-à-vis such models as the Susceptible-Immune-Removed (SIR). In these, the use of chemical sensors or private electronic devices are used, thus causing the need for significant costs, or harming personal privacy.

### SUMMARY OF THE INVENTION

The invention herein is a method to analyze and control the spread of viral, bacterial, fungal or other infections in the form of a pandemic by collecting and analyzing data from different widespread sources. The use of communications, a high number of sensors, the ability to geolocate and geotag measurements, and tools for trend analysis and prediction of large data amounts, allows to detect trends, green-light areas that are not infected, contain infected areas, generate recommendations, analyze the time that it takes to develop symptoms in different demographics and more.

In its preferred embodiment, the invention herein utilizes a large number of thermometers that are typically being used anyhow in a widespread manner in case of a pandemic for screening in specific points, in addition to a connection to some local, remote or both server, in which data is gathered via a communications link. The data gathered can be geotagged by means such as GNSS, or by prior knowledge of the deployment location of a specific device. The gathered data is then gathered, analyzed and presented via the use of GIS systems. Results and recommendations are then presented to a user.

### BRIEF DESCRIPTION OF THE SEVERAL VIEWS OF THE DRAWING(S)

FIG. 1—Schematic representation of communications and control in the preferred embodiment.

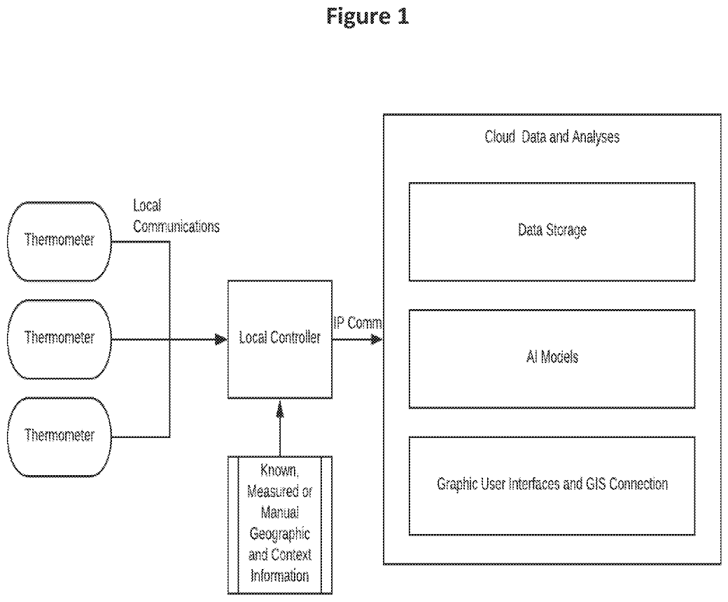

FIG. 2—Schematic representation of the same system utilizing local mobile cellular phones.

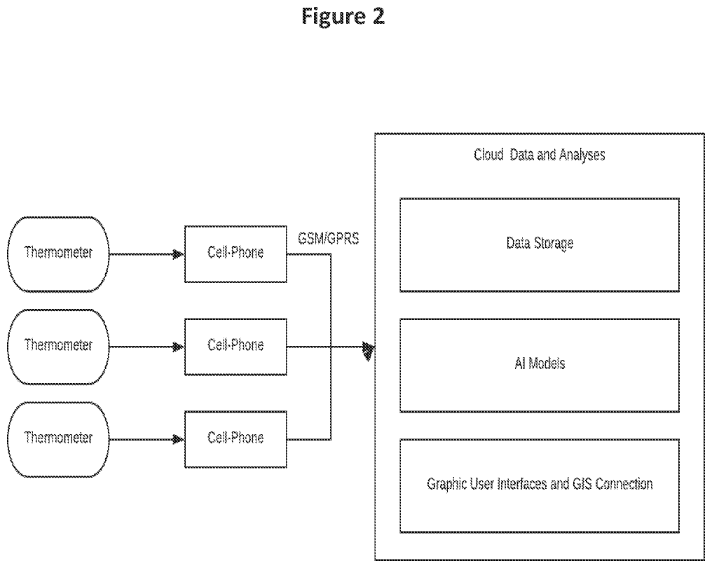

### DESCRIPTION OF SPECIFIC EMBODIMENTS OF THE INVENTION

The invention described herein is a method to control a medical pandemic, usually caused by some pathogen such as a virus, bacterium, fungus, archaeon, mycoplasma or other. In case of a pandemic, in order to limit the spread of the pathogen, different sensors are used to detect people or animals that present a fever or other symptoms. The sensors include, yet are not limited to thermometers, personal declarations of health, microphones, cameras, radars and others. The method described is the use of this multiplicity of sensors and collect the information from them by the usage of common communications protocols and layers to gather the data in a centralized server or data center, and perform useful analyses to allow trend detection and recommendation generation.

In the preferred embodiment, the main sensors in use are thermometers of different types, that are commonly Infra-Red sensors that are aimed at a subject's skin from a close distance to get an indication of fever. Said sensors are typically connected to a communications element such as a Bluetooth transceiver that transmits collected data to a nearby local controller, which can perform initial analyses, pre-processing, and in turn sends the data to centralized server in which all data from all locations is gathered.

In other embodiments, the thermometers can be based solely on other methods, or combine other techniques than Infra-Red. These include laser readings, thermal IR cameras, the use of resistance temperature detectors (RTDs), shape memory materials, thermochromic materials, thermal expansion of materials such as mercury and others. Abovementioned communications can be based solely on Bluetooth protocols, but can alternatively or in addition use such communications protocol as WiFi, Zigbee, NFC, cellular communications such as GSM or GPRS, Bluetooth Low Energy, Z-wave, 6LoWPAN and others. In some embodiments, the local controller is not needed and the end sensors connect directly with the centralized server by one or more of the communications methods mentioned.

The end sensors or the local controllers can be used in conjunction with geolocalization and geotagging tools such as different GNSS systems as GPS and GLONASS, Galileo, Beidou, local RTK stations and others. Geographic data can also be stored manually to reduce costs.

The data gathered from different end-points is typically held in a way that does not harm privacy of the measurement subjects. When transmitted to a central server, it is being used to map geographic and geo-temporal trends. These can be used as input for predictive analyses tools such as neural networks, regression algorithms and others.

The method described herein can also use other sensing capabilities such as the use of physiological measurements as radar measurements to detect breathing patterns, potentially indicating respiratory infections, the use of cameras to detect behavioral changes, use of retro reflection from pupils to assess stress levels or general fatigue by introducing custom devices or by the use of existing common security cameras. Furthermore, other physical sensors can be used such as personal skin conduction measurement (GSR) to assess physiological stress, gait measurement and others. Said sensors can be used in conjunction with personal declarations, questionnaires or other subjective interviewing techniques that can include the use of color-coded results, QR codes coded results, barcode coded results etc.

The sensors in use for data gathering can be ones that are supplied with aforementioned technologies for the sake of pandemics control, or alternatively they can be existing sensors such as thermometers that are already in use and are thus either retrofitted to allow the centralized data collection or used in conjunction with manual data inputting in a phone application, computer or other. One or more of the actions required to send the data, potentially after some type of filtering, can be performed with an operator involved using some interface such as a phone application. Alternatively, all data collection can be performed automatically in such a way like gathering data from a robotic entry control point that requires thermometer measurement.

In such cases as the use of retrofitted devices, a device can be used as part of the method herein to read a screen, grab a result from a galvanic line or connect to a communications port. Said device then retrieves the sensed measurement and transmits it to a local or remote controller or server as described above. The device can include a mechanical component to latch on to an existing device, and can include the same multiplicity of communications mentioned before.

In some embodiments the use of other infection detection measures is used in conjunction with aforementioned sensors. For example, results of genetic testing such as PCR, RT-PCR, qPCR, isothermal amplification or others can be used to cross validate or to add information of infections in specific geographical locations. This can also be used to analyze the correlation between developing symptoms to pandemic spread in different demographics and geographic concentrations locations. The use of serological testing, scent testing or other techniques that can present widespread surveys can further be used together.

In addition this method can also be used to count the number of people in a certain location in a specific time in conjunction with other sensor or sensors that count the number of people leaving the location.

In some embodiments, the use of measures to detect pathogen presence on surfaces as a complementary tool to human infections can be used. In such cases, such tools as materials that respond to the presence of a pathogen can be used, or a direct use of analytical biochemistry tools can be applied. Among other, nano- or micro elements such as quantum dots, nano-particles, liposomes, micelles and others can be engineered to present a viral entry site, or bacterial appropriate media. In such cases, some chemical or physical response can occur such as a spectroscopic detectable response, or the release of a fluorescent material. In all these cases, the data can be transmitted for further analyses as described above.

The applications of the invention herein range from the use to control an ongoing pandemic or epidemic, preventing the spread of an epidemic, detecting the spread of a new pathogen or a potential epidemic in natural circumstances and in cases of biological military attacks or bio-terrorism. It can be used to clear areas that are detected as non-infected to create continuous allowed motion or other types of travel in safety or relative safety from a pathogen.

Furthermore, the invention can serve as a tool for research of a specific contagious disease, or generic research in disease transmission by providing non-privacy-harming information regarding rates and potentially demographics of disease spread with geographic and location-type context. These results or conclusions can also aid in the education and enforcement of measures against the spread of a pandemic, for example by comparing different behaviors in such different locations as a mall, a community center, a worship place, a school and so forth.

### REFERENCES

[1] U.S. Ser. No. 10/520,366B2
[2] WO2011049886A1
[3] U.S. Pat. No. 9,075,909B2

What is claimed is:

1. A system substantially as herein described and illustrated.

2. A method substantially as herein described and illustrated.

3. A computer product for implementing a method substantially as herein described and illustrated.
